(19) United States
(12) Patent Application Publication
(10) Pub. No.: US 2024/0222726 A1
(43) Pub. Date: Jul. 4, 2024

(54) EARLY WARNING METHOD AND SYSTEM FOR DENDRITE GROWTH IN LITHIUM BATTERY

(71) Applicant: SHANGHAI MAKESENS ENERGY STORAGE TECHNOLOGY CO., LTD., Shanghai (CN)

(72) Inventors: Danfei Gu, Shanghai (CN); Qian Li, Shanghai (CN); Mingchen Jiang, Shanghai (CN); Siyuan Chen, Shanghai (CN); Liangchang Wei, Shanghai (CN); Xiao Yan, Shanghai (CN); Enhai Zhao, Shanghai (CN)

(21) Appl. No.: 18/392,122

(22) Filed: Dec. 21, 2023

(30) Foreign Application Priority Data
Dec. 29, 2022 (CN) 202211710801.8

(51) Int. Cl. H01M 10/48; H01M 10/0525

(52) U.S. Cl. CPC H01M 10/48; H01M 10/0525; H01M 2200/00

(57) ABSTRACT

The invention provides a method and a system for early warning of dendrite growth in a lithium battery. The method includes simulating the lithium battery in real time through an electrochemical model to judge whether the lithium battery generates lithium dendrites; simulating a growth trend of the lithium dendrites when the lithium battery is determined to generate the lithium dendrites; and judging and providing early warning of the dendrite growth in the lithium battery based on the growth trend of the lithium dendrites obtained through simulation. The invention prevents the growth of dendrites of the lithium battery by carrying out early warning and simulation for dendrite growth, thereby protecting the safety of the lithium battery system.

### CROSS-REFERENCE TO RELATED PATENT APPLICATION

This application claims priority to and the benefit of Chinese Patent Application No. 202211710801.8, filed Dec. 29, 2022, which is incorporated herein in its entirety by reference.

### FIELD OF THE INVENTION

The invention relates generally to the field of battery management, and more particularly to a method and a system for early warning of dendrite growth in a lithium battery.

### BACKGROUND OF THE INVENTION

In the context of global “carbon neutrality,” enthusiasm for finding clean energy alternatives to petroleum energy continues to rise. Clean and sustainable energy sources such as solar power, tidal energy, wind power, and hydropower are available, but the controllability of the medium generated by these energy sources is relatively weak. Lithium-ion batteries are a new generation of secondary batteries with high energy density and cycle life. They are currently widely used in mobile communications, digital technology, electric vehicles, energy storage, and other fields. The future demand for lithium batteries and their materials is difficult to estimate, and the corresponding upstream and downstream industrial chains also present a huge market.

As the current applications of lithium batteries continue to expand, the safety hazards associated with these batteries are increasingly drawing attention. From mobile phones and laptops to electric vehicles, incidents of lithium-ion batteries heating up or even catching fire have occurred. This phenomenon is no longer limited to counterfeit electronic products. Safety incidents involving lithium-ion batteries have involved brands such as Nikon, Panasonic, Samsung, Xiaomi, Lenovo, Tesla, and others.

Lithium dendrites are one of the fundamental issues affecting the safety and stability of lithium-ion batteries and are an industry pain point. The formation of lithium dendrites can lead to instability at the electrode-electrolyte interface of the lithium-ion battery during cycling. The growth of lithium dendrites can destroy a generated Solid Electrolyte Interface (SEI) film. The lithium dendrites can continuously consume electrolytes in the generation process and cause irreversible deposition of metallic lithium. This results in the formation of dead lithium, leading to low Coulombic efficiency, and can even pierce the separator, causing internal short circuit of the lithium-ion battery. This internal short circuit can trigger thermal runaway, leading to combustion and explosion of the battery.

At present, there is still considerable controversy over the growth mechanism of lithium dendrites, and how to monitor the growth mechanism of lithium dendrites and perform timely early warning has become an urgent problem to be solved.

Therefore, a heretofore unaddressed need exists in the art to address the aforementioned deficiencies and inadequacies.

### SUMMARY OF THE INVENTION

In view of the above-noted shortcomings, one of the objectives of this invention is to provide a method and a system for early warning of dendrite growth in a lithium battery.

In one aspect of the invention, the method includes:
simulating the lithium battery in real time through an electrochemical model to judge whether the lithium battery generates lithium dendrites;
simulating a growth trend of the lithium dendrites when the lithium battery is determined to generate the lithium dendrites; and
judging and providing early warning of the dendrite growth in the lithium battery based on the growth trend of the lithium dendrites obtained through simulation.

In some embodiments, the simulating a growth trend of the lithium dendrites when the lithium battery is determined to generate the lithium dendrites comprises:
coupling growth and stripping formulas of the lithium dendrites into the electrochemical model to obtain a simulation model of a generation direction of the lithium dendrites and a simulation model of a stripping of the lithium dendrites; and
simulating the growth trend of the lithium dendrites based on the simulation model of the generation direction of the lithium dendrites and the simulation model of the stripping of the lithium dendrites.

In some embodiments, the simulating the growth trend of the lithium dendrites based on the simulation model of the generation direction of the lithium dendrites comprises:
simulating based on a phase parameter time-varying function of a phase change point of the generation direction of the lithium dendrites, wherein the phase parameter time-varying function of the phase change point of the generation direction of the lithium dendrites is as follows:

$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$

wherein ∂ξ/∂t is the phase parameter time-varying function of the phase change point of the generation direction of the lithium dendrites, ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ω is an anisotropic mode, θ is a relative angle of an interface normal vector, L_{σ} is an interface migration capability parameter, L_{η} is a forward reaction parameter, F is a Faraday constant, and R is a universal gas constant.

In some embodiments, the simulating the growth trend of the lithium dendrites based on the simulation model of the stripping of the lithium dendrites comprises:
simulating based on a phase parameter time-varying function of a phase change point of the stripping of the lithium dendrites, wherein the phase parameter time-varying function of the phase change point of the stripping of the lithium dendrites is as follows:

$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$

wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration.

In some embodiments, the judging and providing early warning of the dendrite growth in the lithium battery based on the growth trend of the lithium dendrites obtained through simulation comprises:
calculating influence data of the dendrite growth on an electric field based on the growth trend of the lithium dendrites obtained through simulation;
calculating influence data of the dendrite growth on concentration growth based on the growth trend of the lithium dendrites obtained through simulation; and
judging and providing early warning of the dendrite growth in the lithium battery according to the influence data of the dendrite growth on the electric field and the influence data of the dendrite growth on the concentration growth.

In some embodiments, the simulating the lithium battery in real time through an electrochemical model to judge whether the lithium battery generates lithium dendrites comprises:
loading real-time operating condition information of the lithium battery and physical and chemical parameters of the lithium battery into a battery electrochemical model to be simulated; and
performing real-time simulation on the real-time operating information of the lithium battery and the physical and chemical parameters of the lithium battery through the electrochemical model so as to judge whether the lithium battery generates lithium dendrites.

In another aspect, the invention relates to an early warning system for dendrite growth in a lithium battery, comprising:
a judgment module, configured to simulate the lithium battery in real time through an electrochemical model to judge whether the lithium battery generates lithium dendrites;
a simulation module, configured to simulate a growth trend of the lithium dendrites when the lithium battery is determined to generate the lithium dendrites; and
an early warning module, configured to judge and provide early warning of the dendrite growth in the lithium battery based on the growth trend of the lithium dendrites obtained through simulation.

In some embodiments, the simulation module is further configured to:
couple growth and stripping formulas of the lithium dendrites into the electrochemical model to obtain a simulation model of a generation direction of the lithium dendrites and a simulation model of a stripping of the lithium dendrites; and
simulate the growth trend of the lithium dendrites based on the simulation model of the generation direction of the lithium dendrites and the simulation model of the stripping of the lithium dendrites.

In some embodiments, the simulation module is further configured to:
simulate based on a phase parameter time-varying function of a phase change point of the generation direction of the lithium dendrites, wherein the phase parameter time-varying function of the phase change point of the generation direction of the lithium dendrites is as follows:

$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$

wherein ∂ξ/∂t is the phase parameter time-varying function of the phase change point of the generation direction of the lithium dendrites, ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ω is an anisotropic mode, θ is a relative angle of an interface normal vector, L_{σ} is an interface migration capability parameter, L_{η} is a forward reaction parameter, F is a Faraday constant, and R is a universal gas constant.

In some embodiments, the simulation module is further configured to:
simulate based on a phase parameter time-varying function of a phase change point of the stripping of the lithium dendrites, wherein the phase parameter time-varying function of the phase change point of the stripping of the lithium dendrites is as follows:

$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$

wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration.

Compared with the prior art, the method and system for early warning of dendrite growth in a lithium battery provided by the invention can at least bring the following beneficial effects:

The invention prevents the growth of dendrites of the lithium battery by carrying out early warning and simulation for dendrite growth, thereby protecting the safety of the lithium battery system.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 is a flowchart of an early warning method for dendrite growth in a lithium battery according to embodiments of the invention.

FIG. 2 is another flowchart of an early warning method for dendrite growth in a lithium battery according to embodiments of the invention.

FIG. 3 is a schematic diagram of a simulation of a coupling of a lithium dendrite model and an electrochemical model.

FIG. 4 is a schematic diagram of a selected phase parameter time-varying function of a phase change point.

FIG. 5 is a schematic diagram of an early warning system for dendrite growth in a lithium battery according to embodiments of the invention.

### DETAILED DESCRIPTION OF THE INVENTION

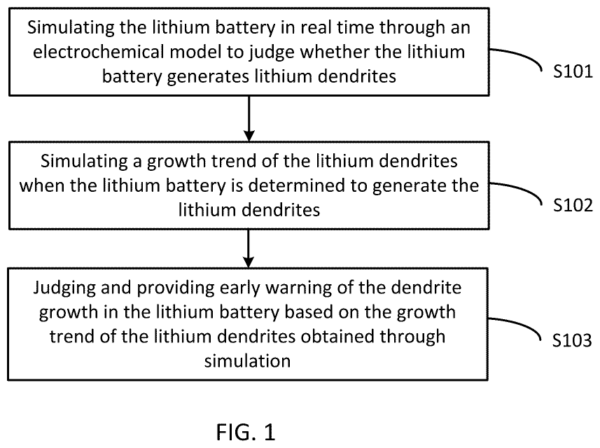

Embodiments of the invention are described below through specific examples in conjunction with the accompanying drawings in FIGS. 1-5, and those skilled in the art can easily understand other advantages and effects of the invention from the content disclosed in this specification. The invention can also be implemented or applied through other different specific implementations, and various modifications or changes can be made to the details in this specification according to different viewpoints and applications without departing from the spirit of the invention. It should be noted that, in the case of no conflict, the following embodiments and features in the embodiments can be combined with each other.

It should be noted that the drawings provided in the following embodiments are merely illustrative in nature and serve to explain the principles of the invention, and are in no way intended to limit the invention, its application, or uses. Only the components related to the invention are shown in the drawings rather than the number, shape and size of the components in actual implementations. For components with the same structure or function in some figures, only one of them is schematically shown, or only one of them is marked. They do not represent the actual structure of the product. Dimensional drawing, the type, quantity and proportion of each component can be changed arbitrarily in its actual implementations. More complicate component layouts may also become apparent in view of the drawings, the specification, and the following claims.

The terminology used herein is for the purpose of describing particular embodiments only and is not intended to be limiting of the invention. As used herein, “a” not only means “only one,” but also means “more than one.” The term “and/or” used in the description of the present application and the appended claims refers to any combination and all possible combinations of one or more of the associated listed items, and includes these combinations. The terms “first,” “second,” etc. are only used for distinguishing descriptions, and should not be construed as indicating or implying relative importance.

It should be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

In order to more clearly illustrate the embodiments of the invention or the technical solutions in the prior art, the following description will explain the specific embodiments of the invention with reference to the accompanying drawings. It is evident that the drawings in the following description are only examples of the invention, from which other drawings and other embodiments can be obtained by a person skilled in the art without inventive effort.

In one embodiment, as shown in FIG. 1, the early warning method for dendrite growth in a lithium battery includes the following steps:

S101, simulating the lithium battery in real time through an electrochemical model to judge whether the lithium battery generates lithium dendrites.

In this embodiment, computational simulation is performed through an electrochemical model based on real-time operating condition information and electrochemical parameters of the lithium battery, and a deposition result of the lithium battery is obtained by analysis. Based on the deposition result of the lithium battery, it is judged whether the lithium battery generates lithium dendrites.

Specifically, the real-time operating condition information and the physical and chemical parameters of lithium battery parameter identification are loaded into a battery electrochemical model to be simulated.

In this embodiment, the electrochemical model simulates the just-loaded parameters in real time.

It should be noted that negative electrode lithium deposition generally occurs before lithium dendrites appear. Lithium dendrites are a particular form of lithium deposition. The growth of lithium deposits changing from mossy lithium to lithium dendrites happens when the mass transfer process cannot keep up with the electrochemical reaction rate, resulting in uneven lithium deposition. At this time, the liquid phase concentration near the lithium deposition is 0 or close to 0, and at this time, the deposition growth guided by the concentration field changes into that guided by the electric field, and the uneven growth form is dendrites.

Before assessing the presence of dendrites, the lithium deposition situation is first considered in the electrochemical model. The electrochemical model of the present invention may not be a full-order or other field coupled electrochemical model, but must be coupled with the influence of lithium deposition.

S102, simulating a growth trend of the lithium dendrites when the lithium battery is determined to generate the lithium dendrites.

In this embodiment, after obtaining the deposition results of the lithium battery, if it is determined that the lithium battery generates lithium dendrites, a growth trend of the lithium dendrites is simulated.

The simulation of the growth trend of the lithium dendrites includes simulation of a generation direction of the lithium dendrites and simulation of a stripping of the lithium dendrites.

S103, judging and providing early warning of the dendrite growth in the lithium battery based on the growth trend of the lithium dendrites obtained through simulation.

In this embodiment, the dendrite growth in each point of the lithium battery is assessed. If a point where a dendrite variable of the lithium battery is not 0 reaches the separator, internal short circuit and electrical breakdown may occur.

Specifically, each point in the computational domain of the lithium battery simulation will have a value. If the value for that point is close to 1, the state at that point is close to a metallic state, that is, dendrite, and if it is close to 0, it is lithium in the electrolyte state.

If a point where the dendrite variable in the lithium battery is 1 reaches the separator, that is, if there is a dendrite variable of 1 at a spatial point near the separator, it can be judged that a physical breakdown or hidden danger exists.

The present invention avoids the growth of dendrites of the lithium battery by providing early warning and simulation for dendrite growth, thereby protecting the safety of the lithium battery system.

In one embodiment, the simulating a growth trend of the lithium dendrites when the lithium battery is determined to generate the lithium dendrites includes:
coupling growth and stripping formulas of the lithium dendrites into the electrochemical model to obtain a simulation model of a generation direction of the lithium dendrites and a simulation model of a stripping of the lithium dendrites; and
simulating the growth trend of the lithium dendrites based on the simulation model of the generation direction of the lithium dendrites and the simulation model of the stripping of the lithium dendrites.

In this embodiment, there is a reversible reaction of lithium dendrite growth and stripping in the lithium dendrite reaction: Li⁺+e⁻Æ Li.

Here, ξ is used to denote the degree of phase evolution of lithium taking part in this reaction somewhere in this space. When ξ is 0, it represents the lithium-ion state completely in the electrolyte, that is, the left side of the chemical reaction equation. The solid phase lithium metal state where Li is a positive electrode represents the solid phase lithium metal state, that is, the right side of the chemical reaction equation. ξ changes from 0 to 1 at the phase change interface, thus characterizing the spatial variation of phase evolution.

Since the overall framework in this embodiment is a numerical simulation framework of an electrochemical model, the research simulation problem is the lithium dendrite problem in lithium batteries, and the growth simulation of lithium dendrites needs to be coupled with the numerical simulation framework of the electrochemical model. According to the following formula, the simulation of embedding the growth-stripping formula of lithium dendrites into the electrochemical model can be obtained.

It should be noted that dendrites in sodium batteries can also be simulated by similar formulas, the difference being that on one hand, the material parameters representing the material properties are different, and on the other hand, the dendrite problem in sodium batteries is not the biggest shortcoming, whereas the dendrite problem in lithium batteries is very serious.

In one embodiment, the simulating the growth trend of the lithium dendrites based on the simulation model of the generation direction of the lithium dendrites includes:
simulating based on a phase parameter time-varying function of a phase change point of the generation direction of the lithium dendrites, wherein the phase parameter time-varying function of the phase change point of the generation direction of the lithium dendrites is as follows:

$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$

wherein ∂ξ/∂t is the phase parameter time-varying function of the phase change point of the generation direction of the lithium dendrites, ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ω is an anisotropic mode, θ is a relative angle of an interface normal vector, L_{σ} is an interface migration capability parameter, L_{η} is a forward reaction parameter, F is a Faraday constant, and R is a universal gas constant.

In one embodiment, the simulating the growth trend of the lithium dendrites based on the simulation model of the stripping of the lithium dendrites includes:
simulating based on a phase parameter time-varying function of a phase change point of the stripping of the lithium dendrites, wherein the phase parameter time-varying function of the phase change point of the stripping of the lithium dendrites is as follows:

$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$

wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration

Specifically, the simulation of the generation direction of the lithium dendrites and the simulation of the stripping of the lithium dendrites involve simulating two opposing reactions. These two reactions ultimately affect the value of ξ at each point in space.

In this embodiment, the formula corresponding to the simulation model of the generation direction of the lithium dendrites and the formula corresponding to the simulation model of the stripping of the lithium dendrites are used to calculate the value of ξ at each point in space. Then, it is judged whether the value of ξ near the separator is close to 1.

In one embodiment, the simulating the lithium battery in real time through an electrochemical model to judge whether the lithium battery generates lithium dendrites includes:
loading real-time operating condition information of the lithium battery and physical and chemical parameters of the lithium battery into a battery electrochemical model to be simulated; and
performing real-time simulation on the real-time operating information of the lithium battery and the physical and chemical parameters of the lithium battery through the electrochemical model so as to judge whether the lithium battery generates lithium dendrites.

In this embodiment, the real-time operating condition information refers to operating condition information for controlling charge and discharge of the battery.

Illustratively, real-time operating condition information includes current-time information, voltage-time information, and power-time information.

In an actual scenario, if the battery is charged and discharged through current control, the real-time operating condition information is current-time information. If the battery is charged and discharged through voltage control, the real-time operating condition information is voltage-time information. If the battery is charged and discharged through power control, the real-time operating condition information is power-time information.

In this embodiment, the electrochemical parameters of the lithium battery refer to the physical and chemical parameters of the lithium battery, and the electrochemical parameters of the lithium battery are the electrochemical parameters of the electrochemical model used.

Computational simulation is performed through an electrochemical model based on real-time operating condition information and electrochemical parameters of the lithium battery, and a deposition result of the lithium battery is obtained by analysis.

Specifically, the real-time operating condition information and the physical and chemical parameters of lithium battery parameter identification are loaded into a battery electrochemical model to be simulated.

In this embodiment, the electrochemical model simulates the just-loaded parameters in real time.

It should be noted that negative electrode lithium deposition generally occurs before lithium dendrites appear. Lithium dendrites are a particular form of lithium deposition. The growth of lithium deposits changing from mossy lithium to lithium dendrites happens when the mass transfer process cannot keep up with the electrochemical reaction rate, resulting in uneven lithium deposition. At this time, the liquid phase concentration near the lithium deposition is 0 or close to 0, and at this time, the deposition growth guided by the concentration field changes into that guided by the electric field, and the uneven growth form is dendrites.

Before assessing the presence of dendrites, the lithium deposition situation is first considered in the electrochemical model. The electrochemical model of the present invention may not be a full-order or other field coupled electrochemical model, but must be coupled with the influence of lithium deposition.

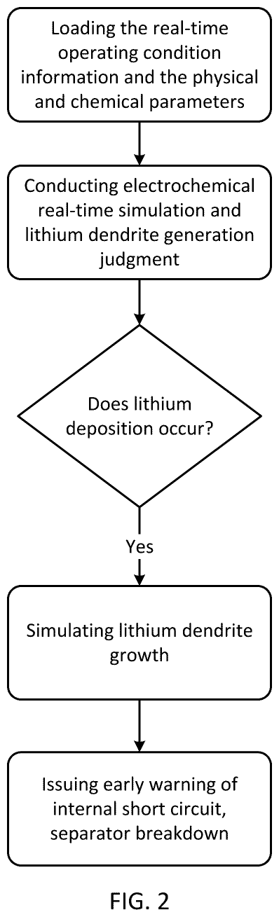

In one embodiment, as shown in FIG. 2, the early warning method for dendrite growth in a lithium battery includes:

The invention provides a numerical simulation and early warning method for dendrite growth in a lithium battery. First, battery operating conditions, parameters, and physical and chemical parameters are loaded. Then, it is judged whether dendrites are formed. After that, dendrite growth is simulated. Finally, dendrites are judged at each point based on the distance from that point to the separator and the length of the dendrites to judge the severity of internal short circuits. A method for establishing a lithium battery module digital twin system includes the following steps:
Step S1, loading parameters

The real-time operating condition information and the physical and chemical parameters of the lithium battery parameter identification are loaded into a battery electrochemical model to be simulated.
Step S2, conducting electrochemical real-time simulation and lithium dendrite generation judgment
Step S3, simulating lithium dendrite growth

There is a reversible reaction of lithium dendrite growth and stripping in the lithium dendrite reaction: Li⁺+e⁻ Æ Li.

In this embodiment, ξ is used to denote the degree of phase evolution of lithium taking part in this reaction somewhere in this space. When ξ is 0, it represents the lithium-ion state completely in the electrolyte, that is, the left side of the chemical reaction equation. 1 represents the solid phase lithium metal state, that is, the right side of the chemical reaction equation. ξ changes from 0 to 1 at the phase change interface, thus characterizing the spatial variation of phase evolution.

Since the overall framework in this embodiment is a numerical simulation framework of an electrochemical model, the research simulation problem is the lithium dendrite problem in lithium batteries, and the growth simulation of lithium dendrites needs to be coupled with the numerical simulation framework of the electrochemical model. According to the following formula, the simulation of embedding the growth-stripping formula of lithium dendrites into the electrochemical model can be obtained. It should be noted that dendrites in sodium batteries can also be simulated by similar formulas, the difference being that on one hand, the material parameters representing the material properties are different, and on the other hand, the dendrite problem in sodium batteries is not the biggest shortcoming, whereas the dendrite problem in lithium batteries is very serious.

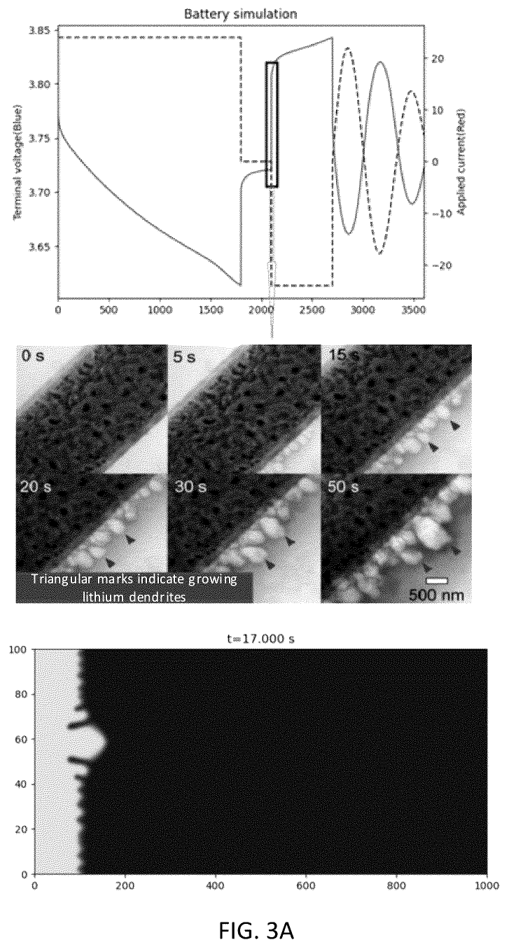

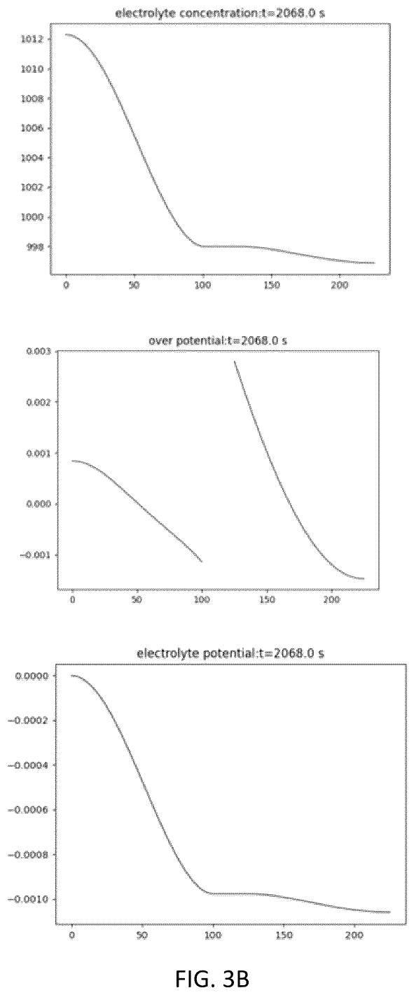

Specifically, as shown in FIG. 3, the simulation in this embodiment is a simulation of the growth of lithium dendrites by coupling a lithium dendrite model with an electrochemical model. The first diagram on the left in FIG. 3 is a voltage simulation diagram of the battery, and the right three diagrams are liquid phase concentration, overpotential, and liquid phase potential diagrams. The second picture on the left shows the actual growth of lithium dendrites, and the third diagram on the left shows a simulated growth.

For the direction of dendrite generation:

$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$

L_{σ} is an interface migration capability parameter, L_{η} is a forward reaction parameter.

Specifically, the calculation of ξ for each point in space is performed based on two PDEs. During visualization, the ξ value is represented by color depth to showcase the growth of lithium dendrites. However, in a computer, the ξ value can be directly used for judgment.

f₀(ξ)=Wξ²(1−ξ)² is an energy barrier functional and can be selected as a characteristic form, where a dual potential well is selected, where W/16 represents the barrier size between two equilibrium states in the electrode and electrolyte. A simpler alternative is f₀=Wξ(1−ξ) or f₀=Wξ ln(1−ξ), which physically represents the difficulty of transition between two phases. In practice, when considering the thermal coupling model, the influence of temperature needs to be taken into account. In this case, W becomes a function of T, and there exists a reference temperature Tc, which is the phase transition point. That is, without needing external stimulus, the potential energy of the two states is equal. An approximate example for the temperature Tc is the phase transition point 0° C. of water and ice. At other temperatures, there will be a tendency for water to more easily become ice or for ice to more easily become water. This temperature is reflected in the functional that appears as

$$\frac{{\partial }^{2} f}{\partial {ϕ}^{2}} = 0 .$$

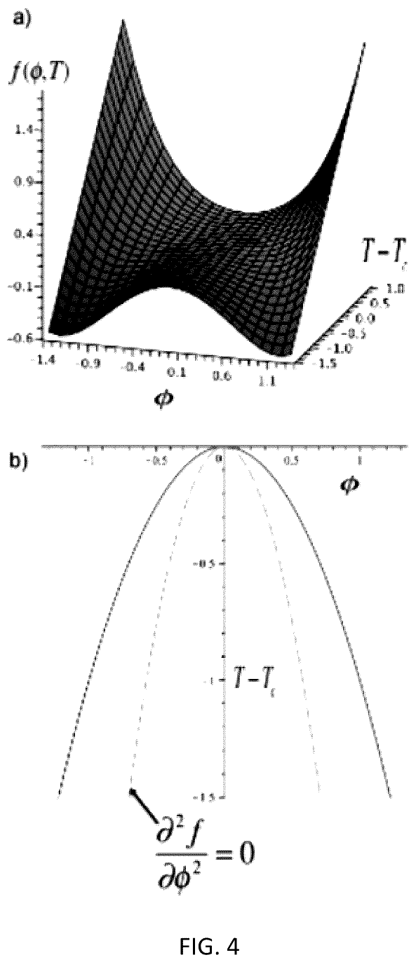

Specifically, Φ in a) shown in FIG. 4 is in the formula, a Landau free energy phase diagram of a simple binary mixture. As shown in b) of FIG. 4, T<Tc is phase-changed from one phase to two phases. The dashed line is the spinodal line.

Looking at the left side, when T−Tc is greater than 0, the lowest point of the isothermal line is one, and when T−Tc is less than 0, it is two. This represents the number of stable phases at different temperatures.

h(ξ)=ξ³(ξ−ξₐ)(ξ−ξ_{b}) is the effect of the concentration field and electric field, characterized by an interpolation function, on dendrite reaction.

α and 1−α are anode-cathode electrical conversion factors.

η=ϕₛ−ϕₑ−U_{eq} is reaction overpotential, ϕₛ is solid phase potential, ϕₑ is liquid phase potential, and U_{eq} is reaction equilibrium voltage. Generally, the reaction equilibrium voltage at normal temperature is 0V, and c_0 is the reference lithium-ion concentration of the electrolyte, generally 1000 mol/L.

fₙₛ(ξ)=h′(ξ)χψ, fₙₛ is a random noise term, χ is a random value from 0-1, ψ is an amplitude. κ can be extended to κ(θ), where κ(θ)=κ_0[1+δ cos ωθ] is related to surface energy anisotropy, κ_0, δ, θ, θ being gradient coefficient, anisotropic strength, anisotropic mode, and relative angle of interface normal vector, respectively.

For the lithium stripping reaction:

$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$

f_{d}=fₛₜₑₚ(−ϕₑ/ϕ_{d}) is an electric field activation state of metallic lithium, ϕ_{d} is a reference potential value, typically set to 1.0 mV in visual models and environmental settings and in reality a function of temperature and pressure, fₛₜₑₚ is a step function, and can also be chosen as a sigmoid function, softmax function, or ReLU function. fd=1 is active metallic lithium, and fd=0 is dead lithium.

α_{Li}_{⁺}=h(c_{Li}_{⁺}/c₀) is a lithium-ion active concentration and h is a function of lithium-ion concentration. Typically, if the lithium-ion concentration is around 1 mol/L or less, h=1. After that, the higher the lithium-ion concentration, the smaller h is, indicating the actual lithium-ion activity level.

In turn, the effect of dendrite growth on the electric field:

$$∇ \cdot ( {σ}^{eff} ∇ ϕ ) = {I}_{R} = n F {c}_{s} \frac{\partial ξ}{\partial t} ;$$

The effect of dendrite growth on the concentration field:

$$\frac{\partial {c}_{{Li}^{+}}}{\partial t} = ∇ \cdot ( {D}_{{Li}^{+}} ( ∇ {c}_{e} + {c}_{e} \frac{F}{R T} ∇ {ϕ}_{e} ) ) - {c}_{s} \frac{\partial ξ}{\partial t}$$

σ^{eff} is an electrical conductivity, D_{Li}_{⁺} is a lithium-ion liquid phase diffusion coefficient, F is a Faraday constant, Ce is a liquid phase lithium-ion concentration, Cs is a solid phase lithium-ion concentration, R is a universal gas constant, T is a Kelvin temperature, and n is a mole number of active substance compound lithium.
Step S4, conducting judgment and early warning of growth

Dendrite growth in each point of the lithium battery is assessed. If a point where a dendrite variable ξ of the lithium battery is not 0 reaches the separator, internal short circuit and electrical breakdown may occur.

If a point where the dendrite variable ξ in the lithium battery is 1 reaches the separator, that is, if there is a dendrite variable of 1 at a spatial point near the separator, it can be judged that a physical breakdown or hidden danger exists.

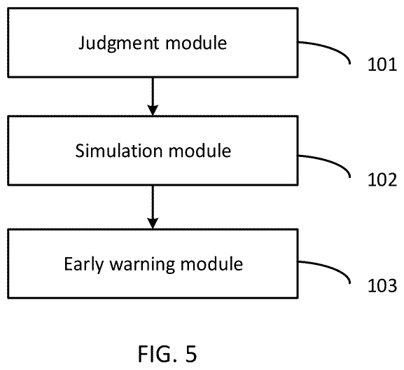

In one embodiment, as shown in FIG. 5, the early warning system for dendrite growth in a lithium battery includes:
a judgment module 101, configured to simulate the lithium battery in real time through an electrochemical model to judge whether the lithium battery generates lithium dendrites;
a simulation module 102, configured to simulate a growth trend of the lithium dendrites when the lithium battery is determined to generate the lithium dendrites; and
an early warning module 103, configured to judge and provide early warning of the dendrite growth in the lithium battery based on the growth trend of the lithium dendrites obtained through simulation.

In one embodiment, the simulation module is further configured to:
simulate based on a phase parameter time-varying function of a phase change point of the generation direction of the lithium dendrites, wherein the phase parameter time-varying function of the phase change point of the generation direction of the lithium dendrites is as follows:

$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$

wherein ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ψ is an anisotropic mode, θ is a relative angle of an interface normal vector, L_{σ} is an interface migration capability parameter, and L_{η} is a forward reaction parameter.

In one embodiment, the simulation module is further configured to:
simulate based on a phase parameter time-varying function of a phase change point of the stripping of the lithium dendrites, wherein the phase parameter time-varying function of the phase change point of the stripping of the lithium dendrites is as follows:

$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$

wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration.

In one embodiment, the judgment module is further configured to:
load real-time operating condition information of the lithium battery and physical and chemical parameters of the lithium battery into a battery electrochemical model to be simulated; and
perform real-time simulation on the real-time operating information of the lithium battery and the physical and chemical parameters of the lithium battery through the electrochemical model so as to judge whether the lithium battery generates lithium dendrites

The invention prevents the growth of dendrites of the lithium battery by carrying out early warning and simulation for dendrite growth, thereby protecting the safety of the lithium battery system.

The foregoing description of the exemplary embodiments of the invention has been presented only for the purposes of illustration and description and is not intended to be exhaustive or to limit the invention to the precise forms disclosed. Many modifications and variations are possible in light of the above teaching. The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. An early warning method for dendrite growth in a lithium battery, comprising:
simulating the lithium battery in real time through an electrochemical model to judge whether the lithium battery generates lithium dendrites;
simulating a growth trend of the lithium dendrites when the lithium battery is determined to generate the lithium dendrites; and
judging and providing early warning of the dendrite growth in the lithium battery based on the growth trend of the lithium dendrites obtained through simulation.

2. The method of claim 1, wherein the simulating a growth trend of the lithium dendrites when the lithium battery is determined to generate the lithium dendrites comprises:
coupling growth and stripping formulas of the lithium dendrites into the electrochemical model to obtain a simulation model of a generation direction of the lithium dendrites and a simulation model of a stripping of the lithium dendrites; and
simulating the growth trend of the lithium dendrites based on the simulation model of the generation direction of the lithium dendrites and the simulation model of the stripping of the lithium dendrites.

3. The method of claim 2, wherein the simulating the growth trend of the lithium dendrites based on the simulation model of the generation direction of the lithium dendrites comprises:
simulating based on a phase parameter time-varying function of a phase change point of the generation direction of the lithium dendrites, wherein the phase parameter time-varying function of the phase change point of the generation direction of the lithium dendrites is as follows:
$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$
wherein ∂ξ/∂t is the phase parameter time-varying function of the phase change point of the generation direction of the lithium dendrites, ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ψ is an anisotropic mode, θ is a relative angle of an interface normal vector, L_{σ} is an interface migration capability parameter, L_{η} is a forward reaction parameter, F is a Faraday constant, and R is a universal gas constant.

4. The method of claim 3, wherein the simulating the growth trend of the lithium dendrites based on the simulation model of the stripping of the lithium dendrites comprises:
simulating based on a phase parameter time-varying function of a phase change point of the stripping of the lithium dendrites, wherein the phase parameter time-varying function of the phase change point of the stripping of the lithium dendrites is as follows:
$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$
wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration.

5. The method of claim 4, wherein the judging and providing early warning of the dendrite growth in the lithium battery based on the growth trend of the lithium dendrites obtained through simulation comprises:
calculating influence data of the dendrite growth on an electric field based on the growth trend of the lithium dendrites obtained through simulation;
calculating influence data of the dendrite growth on concentration growth based on the growth trend of the lithium dendrites obtained through simulation; and
judging and providing early warning of the dendrite growth in the lithium battery according to the influence data of the dendrite growth on the electric field and the influence data of the dendrite growth on the concentration growth.

6. The method of claim 1, wherein the simulating the lithium battery in real time through an electrochemical model to judge whether the lithium battery generates lithium dendrites comprises:
loading real-time operating condition information of the lithium battery and physical and chemical parameters of the lithium battery into a battery electrochemical model to be simulated; and
performing real-time simulation on the real-time operating information of the lithium battery and the physical and chemical parameters of the lithium battery through the electrochemical model so as to judge whether the lithium battery generates lithium dendrites.

7. An early warning system for dendrite growth in a lithium battery, comprising:
a judgment module, configured to simulate the lithium battery in real time through an electrochemical model to judge whether the lithium battery generates lithium dendrites;
a simulation module, configured to simulate a growth trend of the lithium dendrites when the lithium battery is determined to generate the lithium dendrites; and
an early warning module, configured to judge and provide early warning of the dendrite growth in the lithium battery based on the growth trend of the lithium dendrites obtained through simulation.

8. The system of claim 7, wherein the simulation module is further configured to:
couple growth and stripping formulas of the lithium dendrites into the electrochemical model to obtain a simulation model of a generation direction of the lithium dendrites and a simulation model of a stripping of the lithium dendrites; and
simulate the growth trend of the lithium dendrites based on the simulation model of the generation direction of the lithium dendrites and the simulation model of the stripping of the lithium dendrites.

9. The system of claim 8, wherein the simulation module is further configured to:
simulate based on a phase parameter time-varying function of a phase change point of the generation direction of the lithium dendrites, wherein the phase parameter time-varying function of the phase change point of the generation direction of the lithium dendrites is as follows:
$$\frac{\partial ξ}{\partial t} = - {L}_{σ} ( \frac{\partial {f}_{0}}{\partial ξ} - κ {∇}^{2} ξ + \frac{\partial {f}_{n s}}{\partial ξ} ) - {L}_{η} {h}^{′} ( ξ ) [ exp \frac{( 1 - α ) F η}{R T} - \frac{{c}_{{Li}^{+}}}{{c}_{0}} exp \frac{- α F η}{R T} ] ;$$
wherein ∂ξ/∂t is the phase parameter time-varying function of the phase change point of the generation direction of the lithium dendrites, ξ is a phase evolution degree of lithium participating in a growth reaction at a spatial location, t is a current time, T is a current temperature, Li⁺ is a solid phase lithium metal state at a positive electrode, f₀ characterizes a difficulty of transition between two phases, η is a reaction overpotential, c₀ is a reference electrolyte lithium-ion concentration, α and 1−α are anode-cathode electrical conversion factors, fₙₛ(ξ)=h′(ξ)χψ, fₙₛ is a random noise term, χ is a random value from 0-1, ψ is an amplitude, κ is a gradient coefficient, δ is an anisotropic strength, ψ is an anisotropic mode, θ is a relative angle of an interface normal vector, L_{σ} is an interface migration capability parameter, L_{η} is a forward reaction parameter, F is a Faraday constant, and R is a universal gas constant.

10. The system of claim 9, wherein the simulation module is further configured to:
simulate based on a phase parameter time-varying function of a phase change point of the stripping of the lithium dendrites, wherein the phase parameter time-varying function of the phase change point of the stripping of the lithium dendrites is as follows:
$$\frac{\partial ξ}{\partial t} = - {f}_{d} {L}_{σ} ( \frac{\partial {f}_{0}}{\partial ξ} - κ {∇}^{2} ξ + \frac{\partial {f}_{n s}}{\partial ξ} ) - {f}_{d} {L}_{η} h ( ξ ) [ exp \frac{( 1 - α ) F η}{R T} - {α}_{L {i}^{+}} exp \frac{- α F η}{R T} ] ;$$
wherein f_{d}=fₛₜₑₚ(−ϕₑ/ϕ_{d}) is an electric field activation state of metallic lithium, ϕ_{d} is a reference potential value, fₛₜₑₚ is a step function, f_{d} is a state parameter of metallic lithium, α_{Li}_{⁺}=h(c_{Li}_{⁺}/c₀) is a lithium-ion active concentration, and h is a function of lithium-ion concentration.
